(19) United States
(12) Patent Application Publication
(10) Pub. No.: US 2026/0224271 A1
(43) Pub. Date: Aug. 6, 2026

(54) POWER ALLOCATION POLICIES FOR A MULTICHANNEL RADIOFREQUENCY ABLATION SYSTEM

(71) Applicant: Boston Scientific Scimed, Inc., Maple Grove, MN (US)

(72) Inventors: Jin Yu, Valley Village, CA (US); Alexander Pruitt, San Jose, CA (US)

(21) Appl. No.: 19/531,049

(22) Filed: Feb. 5, 2026

Related U.S. Application Data
(60) Provisional application No. 63/754,334, filed on Feb. 5, 2025.

(51) Int. Cl. A61B 18/12; A61B 18/00

(52) U.S. Cl. CPC A61B 18/1206; A61B 2018/00577; A61B 2018/00642; A61B 2018/00678; A61B 2018/00708; A61B 2018/00779; A61B 2018/00875

(57) ABSTRACT

Systems and methods for intelligent power allocation in multichannel radiofrequency (RF) tissue ablation systems are described. The systems include a RF generator with limited power availability coupled to multiple RF probes forming separate channels. When total power demand exceeds generator capacity, the system implements power allocation policies to prevent therapy interruption. These policies include: prioritizing channels based on remaining treatment time, allocating power to channels with higher power demands, using impedance as a power demand predictor, and implementing power consumption thresholds. The controller determines power requirements for each channel and dynamically allocates available power according to the selected policy. This enables controlled treatment completion by intelligently managing limited power resources across multiple channels, avoiding unexpected therapy interruptions that could compromise surgical outcomes. The system can automatically pause, resume, or redistribute power delivery between channels based on real-time demands and priorities during the surgical procedure.

### CROSS REFERENCE TO RELATED APPLICATIONS

This application claims priority to U.S. Provisional Application No. 63/754,334 entitled “POWER ALLOCATION POLICIES FOR A MULTICHANNEL RADIOFREQUENCY ABLATION SYSTEM,” filed Feb. 5, 2025, which is hereby incorporated by reference in its entirety.

### TECHNICAL FIELD

The present invention relates generally to tissue ablation systems and methods of operating the tissue ablation systems. More specifically, the present invention relates to multichannel radiofrequency tissue ablation systems and methods of allocating power delivery in the multichannel radiofrequency tissue ablation systems during surgical procedures.

### BACKGROUND

Radiofrequency energy has been used to ablate tissue at various locations within the body. For example, radiofrequency probes have been used to ablate tissue within the heart to treat atrial fibrillation. Radiofrequency probes have also been used to ablate nerves to facilitate treatment of facet joint pain or joint pain within the knees, shoulders, elbows, hips, or ankles.

A multichannel radiofrequency tissue ablation system includes multiple channels and simultaneous thermal ablations using more than one monopolar or bipolar radiofrequency probe. Multichannel radiofrequency tissue ablation systems with a single radiofrequency generation source have limited power and time resolution, and the stimulation ablation might demand more power than available.

Therefore, there is a need to develop methods to control power delivery and accommodate the amount of power needed to be delivered to each of the channels in multichannel radiofrequency tissue ablation systems with limited power availability.

### SUMMARY

The present invention addresses the need to control power delivery and the amount of power needed to be delivered to each of the channels of a radiofrequency generator by utilizing power allocation policies and methods based on different operational criteria and parameters for prioritizing power delivery to radiofrequency probes coupled to the radiofrequency generator. The power allocation policies enable the system of the present invention to implement a controlled treatment procedure even with limited available power, avoiding unexpected and undesirable interruptions of the therapy process performed by individual radiofrequency probes so that the treatment remains successful as much as reasonably possible.

In one aspect, the system of the present invention provides intelligent power allocation strategies and policies including: fixing priority for power delivery to the most power demanding radiofrequency probe, ordering radiofrequency probes by remaining time to complete therapy, using impedance as a power demand predictor, and setting power consumption thresholds to control initiating ablation on additional radiofrequency probes. The system can dynamically assess and reallocate power based on real-time demands and treatment progress of each channel.

Another aspect of the present invention may include a tissue ablation system comprising a radiofrequency generator having a power source and a controller operatively coupled to the power source and having a processor and a memory element, a first radiofrequency probe having at least one electrode and electrically coupled to the radiofrequency generator to form a first channel, and a second radiofrequency probe having at least one electrode and electrically coupled to the radiofrequency generator to form a second channel. The power source may be configured to deliver radiofrequency power to the one or more electrodes of each probe sufficient to provide controlled heating of tissue surrounding the electrodes during a surgical procedure. The processor executes program instructions to determine power demand required by each channel and, when total power demand exceeds available power, allocate the radiofrequency power according to predetermined allocation policies.

The system implements various power allocation strategies that can include, for example, prioritizing channels based on remaining treatment time, allowing channels with higher immediate power demands to complete therapy first, using measured impedance values to predict and manage power requirements, and employing power consumption thresholds to control when additional probes can begin treatment. These strategies ensure optimal use of available power while maintaining treatment efficacy and preventing unplanned therapy interruptions that could compromise surgical outcomes.

In Example 1, a tissue ablation system is provided. The tissue ablation system comprises a radiofrequency generator having a power source and a controller that is operatively coupled to the power source and having a processor and a memory element. The tissue ablation system further comprises a first radiofrequency probe having at least one electrode and electrically coupled to the radiofrequency generator to form a first channel, and a second radiofrequency probe having at least one electrode and electrically coupled to the radiofrequency generator to form a second channel. The power source can be configured to deliver radiofrequency power to the one or more electrodes of each of the first and second radiofrequency probes. The processor can also be configured to execute program instructions stored in the memory element such that, when executed, causes the controller to determine a power demand required by each of the first channel and the second channel during the surgical procedure, and when a total power demand required by the first and second radiofrequency probes via the first and second channels during the surgical procedure exceeds a total available power of the radiofrequency generator, causing the controller to allocate the radiofrequency power from the radiofrequency generator to one of the first and second channels.

In Example 2, the tissue ablation system of Example 1, wherein the processor can be further configured by the program instructions to cause the controller to prioritize providing the radiofrequency power from the radiofrequency generator to a preselected one of the first and second channels, and cease power supplied to a remaining one of the first and second channels during the surgical procedure.

In Example 3, the tissue ablation system of any of Examples 1-2, wherein the surgical procedure has a first surgical time associated with the first channel and a second surgical time associated with the second channel.

In Example 4, the tissue ablation system of Example 3, wherein the processor is further configured by the program instructions to cause the controller to: compare a first time remaining of the first surgical time in the surgical procedure associated with the first channel with a second time remaining of the second surgical time in the surgical procedure associated with the second channel, and based on the comparison, provide the radiofrequency power from the radiofrequency generator to one of the first channel and the second channel having a lesser amount of time remaining in the surgical procedure.

In Example 5, the tissue ablation system of Example 4, wherein the processor is further configured by the program instructions to cause the controller to order the first and second channels based on the amount of time remaining in the surgical procedure.

In Example 6, the tissue ablation system of any of Examples 1-5, wherein the surgical procedure has a total power demand associated therewith and wherein the first channel has a first power demand associated therewith during the surgical procedure and the second channel has a second power demand associated therewith during the surgical procedure.

In Example 7, the tissue ablation system of Example 6, wherein the processor is further configured by the program instructions to cause the controller to: compare the first power demand with the second power demand, and provide the radiofrequency power from the radiofrequency generator to one of the first and second channels having the greater power demand to complete the surgical procedure to form a prioritized channel.

In Example 8, the tissue ablation system of Example 7, the processor is further configured by the program instructions to cause the controller to: determine an average power demand of the prioritized channel, and if the average power demand of the prioritized channel is above a power demand threshold, then disable an ability to provide radiofrequency power from the radiofrequency generator to a remaining one of the first and second channels.

In Example 9, the tissue ablation system of any of Examples 7-8, wherein the processor is further configured by the program instructions to cause the controller to: reassess the first and second power demands of the first and second channels once the prioritized channel completes the surgical procedure, and if the power demand of any one of the first and second channels exceeds the total available power of the radiofrequency generator: reset priority to the channel having a power demand that is less than or equal to the total available power of the radiofrequency generator to form a new prioritized channel, and cause the power source to deliver radiofrequency power to the new prioritized channel while pausing or disallowing radiofrequency power delivery to the remaining one of the first and second channels until the new prioritized channel completes therapy.

In Example 10, the tissue ablation system of any of Examples 1-9, wherein the processor is further configured by the program instructions to cause the controller to: determine a first impedance associated with the first channel and a second impedance associated with the second channel, and compare the first impedance with the second impedance, and provide the radiofrequency power from the radiofrequency generator to one of the first and second channels having a higher impedance amount.

In Example 11, the tissue ablation system of any of Examples 1-10, wherein the processor is further configured by the program instructions to cause the controller to order the first and second channels based on time remaining for completing the surgical procedure.

In Example 12, the tissue ablation system of any of Examples 1-11, wherein the radiofrequency power is sufficient to provide controlled heating of tissue surrounding the one or more electrodes during a surgical procedure.

In Example 13, the tissue ablation system of any of Examples 1-12, wherein at least one of the first radiofrequency probe and second radiofrequency probe further comprises a temperature sensor.

In Example 14, the tissue ablation system of any of Examples 1-13, wherein at least one of the first radiofrequency probe and the second radiofrequency probe is a bipolar radio frequency probe.

In Example 15, the tissue ablation system of any of Examples 1-14, wherein at least one of the first radiofrequency probe and the second radiofrequency probe is a monopolar radio frequency probe.

In Example 16, a tissue ablation system is provided. The tissue ablation system comprises a radiofrequency generator having a power source and a controller that is operatively coupled to the power source and having a processor and a memory element. The tissue ablation system further comprises a first radiofrequency probe having at least one electrode and electrically coupled to the radiofrequency generator to form a first channel, and a second radiofrequency probe having at least one electrode and electrically coupled to the radiofrequency generator to form a second channel. The power source can be configured to deliver radiofrequency power to the one or more electrodes of each of the first and second radiofrequency probes, the radiofrequency power being sufficient to provide controlled heating of tissue surrounding the one or more electrodes during a surgical procedure. The processor can also be configured to execute program instructions stored in the memory element such that, when executed, causes the controller to determine a power demand required by each of the first channel and the second channel during the surgical procedure, and when a total power demand required by the first and second radiofrequency probes via the first and second channels during the surgical procedure exceeds a total available power of the radiofrequency generator, causing the controller to allocate the radiofrequency power from the radiofrequency generator to one of the first and second channels.

In Example 17, the tissue ablation system of Example 16, wherein the processor is further configured by the program instructions to cause the controller to: prioritize providing the radiofrequency power from the radiofrequency generator to a preselected one of the first and second channels, and cease power supplied to a remaining one of the first and second channels during the surgical procedure.

In Example 18, the tissue ablation system of Example 16, wherein the surgical procedure has a first surgical time associated with the first channel and a second surgical time associated with the second channel, and wherein the processor is further configured by the program instructions to cause the controller to: compare a first time remaining of the first surgical time in the surgical procedure associated with the first channel with a second time remaining of the second surgical time in the surgical procedure associated with the second channel, and based on the comparison, provide the radiofrequency power from the radiofrequency generator to one of the first channel and the second channel having a lesser amount of time remaining in the surgical procedure.

In Example 19, the tissue ablation system of Example 18, wherein the processor is further configured by the program instructions to cause the controller to order the first and second channels based on the amount of time remaining in the surgical procedure.

In Example 20, the tissue ablation system of Example 16, wherein the surgical procedure has a total power demand associated therewith and wherein the first channel has a first power demand associated therewith during the surgical procedure and the second channel has a second power demand associated therewith during the surgical procedure, wherein the processor is further configured by the program instructions to cause the controller to: compare the first power demand with the second power demand, and provide the radiofrequency power from the radiofrequency generator to one of the first and second channels having the greater power demand to complete the surgical procedure to form a prioritized channel.

In Example 21, the tissue ablation system of Example 20, wherein the processor is further configured by the program instructions to cause the controller to: determine an average power demand of the prioritized channel, and if the average power demand of the prioritized channel is above a power demand threshold, then disable an ability to provide radiofrequency power from the radiofrequency generator to a remaining one of the first and second channels.

In Example 22, the tissue ablation system of Example 20, wherein the processor is further configured by the program instructions to cause the controller to: reassess the first and second power demands of the first and second channels once the prioritized channel completes the surgical procedure, and if the power demand of any one of the first and second channels exceeds the total available power of the radiofrequency generator: reset priority to the channel having a power demand that is less than or equal to the total available power of the radiofrequency generator to form a new prioritized channel, and cause the power source to deliver radiofrequency power to the new prioritized channel while pausing or disallowing radiofrequency power delivery to the remaining one of the first and second channels until the new prioritized channel completes therapy.

In Example 23, the tissue ablation system of Example 16, wherein the processor is further configured by the program instructions to cause the controller to: determine a first impedance associated with the first channel and a second impedance associated with the second channel, and compare the first impedance with the second impedance, and provide the radiofrequency power from the radiofrequency generator to one of the first and second channels having a higher impedance amount.

In Example 24, the tissue ablation system of Example 16, wherein the processor is further configured by the program instructions to cause the controller to order the first and second channels based on time remaining for completing the surgical procedure.

In Example 25, a multichannel radiofrequency tissue ablation system is provided, The system comprises a radiofrequency generator having a power source and a controller operatively coupled to the power source and having a processor and a memory element. The system further comprises a first radiofrequency probe having at least one electrode and is electrically coupled to the radiofrequency generator to form a first channel, and a second radiofrequency probe having at least one electrode and is electrically coupled to the radiofrequency generator to form a second channel. The power source is also configured to deliver radiofrequency power to the one or more electrodes of each of the first and second radiofrequency probes, the radiofrequency power being sufficient to provide controlled heating of tissue surrounding the one or more electrodes during a surgical procedure. The controller also includes a power determination and allocation unit to determine a power demand required by each of the first channel and the second channel during the surgical procedure, and when a total power demand required by the first and second radiofrequency probes via the first and second channels during the surgical procedure exceeds a total available power of the radiofrequency generator, the power determination and allocation unit allocates the radiofrequency power from the radiofrequency generator to one of the first and second channels.

In Example 26, the system of Example 25, wherein the power determination and allocation unit is further configured to prioritize providing the radiofrequency power from the radiofrequency generator to a preselected one of the first and second channels, and cease power supplied by the radiofrequency generator to a remaining one of the first and second channels during the surgical procedure.

In Example 27, the system of Example 25, wherein the surgical procedure has a first surgical time associated with the first channel and a second surgical time associated with the second channel, and wherein the controller further comprises a comparison unit for comparing a first time remaining of the first surgical time in the surgical procedure associated with the first channel with a second time remaining of the second surgical time in the surgical procedure associated with the second channel, and wherein, based on the comparison, the power determination and allocation unit is configured to enable the radiofrequency generator to supply the radiofrequency power to one of the first channel and the second channel.

In Example 28, the system of Example 25, wherein the surgical procedure has a total power demand associated therewith and wherein the first channel has a first power demand associated therewith during the surgical procedure and the second channel has a second power demand associated therewith during the surgical procedure, and wherein the controller further comprises a comparison unit for comparing the first power demand with the second power demand, and wherein, based on the comparison, the power determination and allocation unit is configured to enable the radiofrequency generator to supply the radiofrequency power to one of the first and second channels having the greater power demand to complete the surgical procedure to form a prioritized channel.

In Example 29, the system of Example 28, wherein the power determination and allocation unit is further configured to determine an average power demand of the prioritized channel, and if the average power demand of the prioritized channel is above a power demand threshold, then disable an ability to for the radiofrequency generator to provide radiofrequency power to a remaining one of the first and second channels.

In Example 30, the system of Example 25, wherein the controller further comprises an impedance determination unit to determine a first impedance associated with the first channel and a second impedance associated with the second channel, wherein the comparison unit is configured to compare the first impedance with the second impedance, and wherein, based on the comparison, the power determination and allocation unit is configured to enable the radiofrequency generator to provide the radiofrequency power to one of the first and second channels having a higher impedance amount.

In Example 31, a compute implemented method of allocating power delivery in a tissue ablation system is provided. The system comprises: a radiofrequency generator having a power source and a controller operatively coupled to the power source and having a processor and a memory element, a first radiofrequency probe having at least one electrode and electrically coupled to the radiofrequency generator to form a first channel, and a second radiofrequency probe having at least one electrode and electrically coupled to the radiofrequency generator to form a second channel. The power source can be configured to deliver radiofrequency power to the one or more electrodes of each of the first and second radiofrequency probes, the radiofrequency power being sufficient to provide controlled heating of tissue surrounding the one or more electrodes during a surgical procedure. The method comprises executing via the processor program instructions stored in the memory element to: cause the controller to determine a power demand required by each of the first channel and the second channel during the surgical procedure, and when a total power demand required by the first and second radiofrequency probes via the first and second channels during the surgical procedure exceeds a total available power of the radiofrequency generator, cause the controller to allocate the radiofrequency power from the radiofrequency generator to one of the first and second channels.

In Example 32, the computer-implemented method of Example 31, wherein the surgical procedure has a first surgical time associated with the first channel and a second surgical time associated with the second channel, and wherein the method further comprises executing via the processor program instructions stored in the memory element to: compare a first time remaining of the first surgical time in the surgical procedure associated with the first channel with a second time remaining of the second surgical time in the surgical procedure associated with the second channel, and provide the radiofrequency power from the radiofrequency generator to one of the first channel and the second channel having a lesser amount of time remaining in the surgical procedure.

In Example 33, the computer-implemented method of Example 31, wherein the surgical procedure has a total power demand associated therewith and wherein the first channel has a first power demand associated therewith during the surgical procedure and the second channel has a second power demand associated therewith during the surgical procedure, and wherein the method further comprises executing via the processor program instructions stored in the memory element to: compare the first power demand with the second power demand, and provide the radiofrequency power from the radiofrequency generator to one of the first and second channels having the greater power demand to complete the surgical procedure to form a prioritized channel.

In Example 34, the computer-implemented method of Example 33, further comprising executing via the processor program instructions stored in the memory element to: determine an average power demand of the prioritized channel, and if the average power demand of the prioritized channel is above a power demand threshold, then disable an ability to for the radiofrequency generator to provide radiofrequency power to a remaining one of the first and second channels.

In Example 35, the computer-implemented method of Example 31, further comprising executing via the processor program instructions stored in the memory element to:
determine a first impedance associated with the first channel and a second impedance associated with the second channel, and compare the first impedance with the second impedance, and provide the radiofrequency power from the radiofrequency generator to one of the first and second channels having a higher impedance amount.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic block diagram of an exemplary multichannel radiofrequency tissue ablation system, in accordance with the teachings of the present invention;

FIG. 2 is a schematic block diagram of the controller of the multichannel radiofrequency tissue ablation system of FIG. 1 in accordance with the teachings of the present invention;

FIG. 3 is a schematic flow chart diagram illustrating a method of allocating power delivery to channels of the multichannel radiofrequency tissue ablation system with limited power availability during a surgical procedure, in accordance with the teachings of the present invention;

FIG. 4 is a schematic flow chart diagram illustrating an embodiment of the power allocation method of FIG. 3 according to the teachings of the present invention;

FIG. 5 is a schematic flow chart diagram illustrating another embodiment of the power allocation method of FIG. 3 according to the teachings of the present invention;

FIG. 6 is a schematic flow chart diagram illustrating yet another embodiment of the power allocation method of FIG. 3 according to the teachings of the present invention; and

FIG. 7 is a schematic flow chart diagram illustrating a method of allocating power delivery to channels of a multichannel radiofrequency tissue ablation system with limited power availability during a surgical procedure according to the teachings of the present invention.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

A radiofrequency ablation (RFA) procedure is a minimally invasive medical procedure that is used to ablate targeted tissue at a surgical site, and typically involves using one or more energy delivery devices (e.g., radiofrequency (RF) probes) having one or more electrodes associated therewith. The radiofrequency probe can be coupled to one or more radiofrequency (RF) generators to deliver power in the form of radiofrequency energy to the targeted tissue via the electrodes. The radiofrequency energy generates localized heat which is used to target and destroy (e.g., ablate) the targeted tissue. In pain management, the targeted tissue can be nerve tissue responsible for sending pain signals.

The RFA procedure can be utilized to ablate different types of tissue, such as nerve tissue, adipose tissue, muscle tissue, and the like. The RFA procedure can be performed at various locations on an external surface of a subject or patient or within the subject. The RFA procedure performed within the subject can be performed in a minimally invasive manner (e.g., via a percutaneous, laparoscopic, endoscopic, or intravascular approach) or in a more invasive manner (e.g., via an open surgical procedure). For example, Applicant's existing technology (the Intracept® procedure by Relievant® Medsystems, Inc.) offers a safe and effective minimally invasive procedure that targets the basivertebral nerve (BVN) for the relief of chronic low back pain that originates at least partly from one or more endplates of one or more vertebral bodies. The Intracept® procedure involves application of radiofrequency energy from a radiofrequency generator using a bipolar radiofrequency probe, where the applied radiofrequency energy is sufficient to ablate the basivertebral nerve within the vertebral body. The procedure can be performed in multiple different vertebral bodies sequentially or simultaneously using a single radiofrequency probe or using multiple radiofrequency probes coupled to a single generator. The basivertebral nerve trunk can then be ablated by the RF probe. Other intraosseous nerves within the vertebral body that innervate the endplates and/or intervertebral disc can also be targeted and ablated.

The RFA procedure is typically performed using a surgical introducing or access assembly that can include a hollow cannula that forms a sheath and a stylet that is disposed within the cannula until a distal tip of the stylet extends beyond the cannula. The stylet adds rigidity and support to the cannula when inserted into the patient. The stylet helps the surgeon guide the assembly to the surgical site. Once the cannula is in position at the surgical site, then the stylet is removed from the cannula and the RF probe is inserted therein. The RF probe can then deliver RF energy to the targeted tissue at the surgical site. In a bipolar RF probe arrangement, the probe can include a pair of electrodes, including a first active energy delivering electrode and a second return electrode. According to another arrangement, the RF probes can be configured in a monopolar arrangement, where an RF probe employs an active energy delivering electrode and a separate device or element can be used as a return. The return can be configured as a return pad coupled to the subject or a return electrode disposed on a separate RF probe. For example, a first RF probe can be used that includes the active energy delivering electrode and a second RF probe can be used that includes a return electrode. The RF probe can also include additional structure, including for example temperature sensors (e.g., thermocouples or thermistors), that can be employed to sense or detect the temperature at the surgical site during the surgical procedure. The sensed or detected temperature can provide important information to the surgeon about the ablation procedure. The RFA procedure contemplates positioning the two electrodes of the one or more radiofrequency probes within the vertebral body. The RF generator supplies power to the active electrode of the RF probe at a selected power or voltage level and for a time sufficient to create a desired lesion within the vertebral body sufficient to ablate the basivertebral nerve within the vertebral body. In some implementations, the step of applying power to the RF probe includes supplying power for a selected duration or period of time (e.g., 1 minute to 1 hour or for any time within this range) to achieve a selected temperature at the surgical site. The temperature of the thermal energy supplied by the RF probe to the targeted tissue can range from between about 70 and about 115 degrees Celsius (e.g., from about 70 to about 90 degrees Celsius, from about 75 to about 90 degrees Celsius, from about 83 to about 87 degrees Celsius, from about 80 to about 100 degrees Celsius, from about 85 to about 95 degrees Celsius, from about 90 to about 110 degrees Celsius, from about 95 to about 115 degrees Celsius, or overlapping ranges thereof). The temperature ramp up during the surgical procedure can range from between about 0.1-5 degrees Celsius/second (e.g., 0.1-1.0 degrees Celsius/second, 0.25 to 2.5 degrees Celsius/second, 0.5-2.0 degrees Celsius/second, 1.0-3.0 degrees Celsius/second, 1.5-4.0 degree Celsius/second, 2.0-5.0 degrees Celsius/second). The time of treatment can range from between about 10 seconds and about 1 hour (e.g., from 10 seconds to 1 minute, 1 minute to 5 minutes, from 5 minutes to 10 minutes, from 5 minutes to 20 minutes, from 8 minutes to 15 minutes, from 10 minutes to 20 minutes, from 15 minutes to 30 minutes, from 20 minutes to 40 minutes, from 30 minutes to 1 hour, from 45 minutes to 1 hour, or overlapping ranges thereof). Pulsed energy can be delivered as an alternative to or in sequence with continuous energy. For radiofrequency energy, the energy applied by the RF generator can range from between about 350 kHz and about 650 kHz (e.g., from 400 kHz to 600 kHz, from 350 kHz to 500 kHz, from 450 kHz to 550 kHz, from 500 kHz to 650 kHz, overlapping ranges thereof, or any value within the recited ranges, such as 450 kHz±5 kHz, 475 kHz±5 kHz, 487 kHz±5 kHz). The power of the radiofrequency energy generated by the RF generator can range from between about 5 W and about 30 W (e.g., from 5 W to 15 W, from 5 W to 20 W, from 8 W to 12 W, from 10 W to 25 W, from 15 W to 25 W, from 20 W to 30 W, from 8 W to 24 W, and overlapping ranges thereof, or any value within the recited ranges).

Although described primarily in connection with procedures for the treatment within the spine, the systems and methods of the present invention can also be used for radiofrequency tissue ablation procedures intended to treat ailments or conditions other than those associated with the spine or back pain. The procedures can involve ablation of nerves outside of bones but related to the spine (e.g., sacroiliac joints, facet joints, etc.). Target treatment locations within bones other than vertebral bodies may also be accessed. For example, target treatment locations within a humerus, radius, femur, tibia, calcaneus, tarsal bones, hips, knees, phalanges, and/or other orthopedic targets can be accessed. The ablation procedures can include, for example, ablation of nerves within or surrounding other bones other than the vertebral column, cardiac tissue ablation for treatment of atrial fibrillation or other abnormal heart rhythms irregularities, tumor ablation at any location within the body (e.g., within bones, lungs, breasts, thyroids, livers, or other organs or tissues), peripheral nerve ablation, pulmonary artery ablation, renal denervation procedures, uterine fibroid ablation, endometrial ablation, and/or the like. The systems and methods described herein may be used in connection with any radiofrequency procedure during which impedance (e.g., tissue impedance or impedance between two electrodes) is monitored.

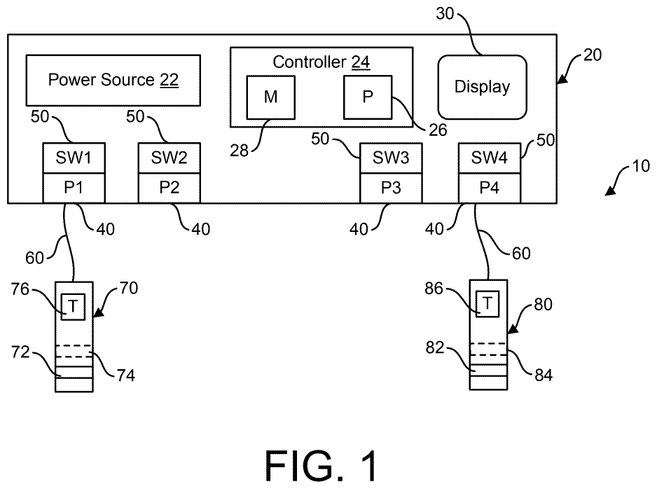

FIG. 1 is a schematic block diagram of a tissue ablation system 10 suitable for use with the present invention. The tissue ablation system 10 includes a generator, such as a radiofrequency generator 20, that is coupled to one or more radiofrequency probes. The RF generator 20 can be electrically and communicatively coupled to the RF probes 70, 80 via any suitable conduit 60. The conduits 60 can facilitate bidirectional communication of electrical signals and data or instructions between the RF generator 20 and the RF probes 70, 80. For example, the conduits 60 can include one or more electrical wires or lines (not shown) suitable for conveying the electrical signals. The generator can also be any other type of energy source suitable for supplying ablative energy to the probes.

According to one embodiment, the illustrated RF generator 20 can include a power source 22 that can be configured to generate and supply radiofrequency power or energy to the RF probes 70, 80 via the conduits 60 at a desired frequency and power level. The RF generator 20 can also include a controller 24 that includes for example a processor (e.g., CPU) 26 suitable for processing and executing applications and instructions that are stored in a memory element 28. The processor 26 can be any suitable type of processor, such as a special purpose processor, and the memory element 28 can be any suitable type of memory. The processor 26 can be implemented in hardware, software, firmware, or any suitable combination thereof, and can be configured to execute program instructions stored in the memory element 28. The processor 26 and the memory element 28 are shown herein as forming part of the controller 24, but one of ordinary skill in the art will readily recognize that the processor 26 and the memory 28 can form part of other components of the RF generator 20. For the sake of simplicity, only one processor 26 and memory element 28 are shown, although multiple processors and memory elements can also be employed in the RF generator 20. The controller 24 can be configured as described herein, and at least is configured to monitor temperature, power, and/or impedance in connection with a tissue ablation procedure performed by the tissue ablation system 10. The programming instructions can be implemented in C, C++, JAVA, or any other suitable programming languages. In some embodiments, some or all of the portions of the controller 24 can be implemented in application specific circuitry such as ASICs and FPGAs.

The RF generator 20 can further include a user interface or display element 30 suitable for displaying information or instructions to a user (e.g., surgeon) via a series of user interfaces or for allowing the user to provide instructions to the RF generator. As such, the display element 30 can employ a user interface generator (not shown) for generating the user interfaces based on programmatic instructions stored in the memory element 28 and processed by the processor 28 or by a processor forming part of the display element 30. The display element 30 can be configured to display information to the user. For example, during startup and use, the current status of the RF generator 20 and energy delivery or treatment parameters can be displayed on the display element 30. During energy delivery, the display element 30 can be configured to display desired treatment time, remaining treatment time, temperature, impedance, and power information (alphanumerically and/or graphically). For example, graphical representations of power vs. time and impedance vs. time can be displayed. The display element 30 can also be separate from the RF generator 20 and need not form part of the generator. Although not shown, the tissue ablation system 10 can also employ additional accessory devices, such as suitable user input devices including a keyboard, mouse, trackpad, voice-activated input device, and the like.

The illustrated RF generator 20 can also have formed therein a plurality or series of connector ports 40 that allow the RF probes 70, 80 to be coupled to the generator. The RF generator 20 can have any selected number of connector ports 40. In the illustrated embodiment, the RF generator 20 includes four connector ports 40. The connector ports 40 can have any selected size, shape, and configuration. According to one embodiment, the connector ports 40 can employ a series of electrical pins that connect to RF probes and allow electrical energy and data to be exchanged therebetween. For example, a first electrical pin (or multiple electrical pins) can be coupled to the RF probe so as to deliver RF power to an energy delivering electrode when placed thereon and a second electrical pin can communicate with a return electrode when placed thereon. A third electrical pin can be employed to communicate with a temperature sensor in the RF probe and a fourth electrical pin can be used to exchange data between the RF generator and the RF probe. Those of ordinary skill in the art will readily recognize that any suitable type (e.g., Lemo or Din type connectors), arrangement, and number of electrical connectors can be employed in the connector ports 40.

The illustrated RF generator 20 can also employ a switching element module or a series of discrete switching elements 50 that can be used to help control the generation, modulation, and delivery of RF energy. The switching elements 50 help ensure the precise delivery of energy to the target tissue via the RF probes while maintaining system safety and functionality. The switching elements 50 can also control the RF energy delivered to the active energy delivering electrode of the RF probe through an associated connector port 40. For the sake of simplicity, a switching element 50 is shown coupled to and disposed in electrical communication with each illustrated connector port 40. Those of ordinary skill in the art will readily recognize that a separate switching element module that is arranged in electrical communication with each of the connector ports 40 can also be employed. The illustrated switching elements 50 can also be positioned and arranged to manage the activation of circuits connected to sensors in the RF probes 70, 80, such as temperature sensors or impedance measurement systems. The switching elements can also be arranged to route sensor signals to specific monitoring circuits in the controller 24 for processing. The switching elements 50 can also serve to verify proper grounding or return electrode connection before enabling RF output to ensure patient safety. The switching elements 50 can be any suitable type of switching element, such as MOSFETs, IGBTs, Pin diodes, mechanical and solid state relays, and the like.

The illustrated tissue ablation system 10 can also employ two or more RF probes. The RF probes can be the same or can be different depending upon the selected type of surgical procedure being performed and/or the purpose and function of the RF probe. The RF probe 70 has a main body having an outer surface having one or more electrodes mounted thereon. In a bipolar RF probe arrangement, the RF probe 70 can have a first active energy delivering electrode 72 and a return electrode 74 disposed on an outer surface of the main body. The probe can also include a temperature sensor 76 for sensing or measuring temperature at the surgical site. The electrodes and the temperature sensor 76 can be coupled to the RF generator 20 via electrical leads that pass through the conduit 60. If the RF probe 70 is constructed as a monopolar device, then the probe can include a single active electrode 72, which can function as the active energy delivering electrode or as the return electrode. In this type of arrangement, the second electrode 74 (in dashed lines) is either inactivated or not present. Similarly, the RF probe 80 has a main body having an outer surface having one or more electrodes mounted thereon. In a bipolar RF probe arrangement, the RF probe 80 can also have a first active energy delivering electrode 82 and a return electrode 84. The probe can also include a temperature sensor 86. The electrodes and the sensor can also be coupled to the RF generator 20 via electrical leads that pass through the conduit 60. If the RF probe 80 is constructed as a monopolar device, then the probe can also include a single active electrode 82, which can function as the active energy delivering electrode or as the return electrode. In this type of arrangement, the second electrode 84 is either inactivated or not present. Each RF probe that is coupled to a connector port 40 of the RF generator 20 forms in essence a channel. As such, multiple RF probes coupled to multiple connector ports 40 of the RF generator 20 can form multiple channels.

The systems and methods described herein are generally aimed at providing power allocation approaches to prioritize power availability in the channels of multichannel radiofrequency tissue ablation systems with a single radiofrequency generation source having limited power and time resolution, in which the stimulation ablation might demand more power than available. Energy demand might be particularly challenging during radiofrequency thermal ramping period (initial therapy ramp, ILM ramp, ramp after high impedance event). The power allocation policies must ensure that the following requirement is satisfied:

$${\sum }_{k = 0}^{n} P o w e r C {h}_{k} \leq Total Available Power$$

where PowerChₖ is an amount of power demanded by a k^{th} channel, n is the total number of channels, which power demand amounts are summarized, Total Available Power is a total power available for delivery to the channels by the RF generator.

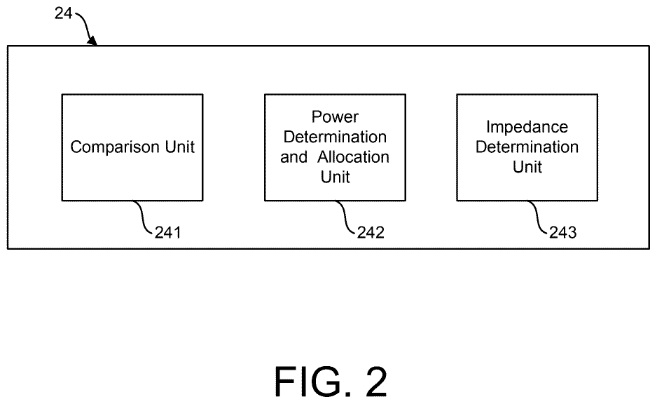

For implementing methods of allocation of power delivery in the multichannel radiofrequency tissue ablation system 10 at limited power availability, the controller 24 can include a comparison unit 241, a power determination and allocation unit 242, and an impedance determination unit 243, as shown in FIG. 2. The comparison unit 241 can form part of the illustrated power determination and allocation unit 242 and/or the impedance determination unit 243 rather than be a separate component or unit. For the sake of simplicity and the ease of discussion, the comparison unit 241 is shown as a separate unit. The power determination and allocation unit 242 of the controller 24 can be configured to determine power demand required by each of the channels during the surgical procedure and allocate the radiofrequency power from the generator 20 to one or more of the RF probes 70, 80 based on preset power allocation policies as disclosed herein. The impedance determination unit 243 of the controller 24 can be configured to determine channel impedance as a power demand predictor for the purpose of determining power demand and allocating power delivery to one or more of the channels as disclosed herein. In some embodiments, the comparison unit 241 of the controller 24 can be configured to compare power demands of the channels, impedances of the channels, and/or time remaining to complete a surgical procedure by one or more of the channels, for the purpose of allocating power delivery to one or more of the channels as disclosed herein. The controller 24 can also be configured to order or select the channels based on priority for power delivery.

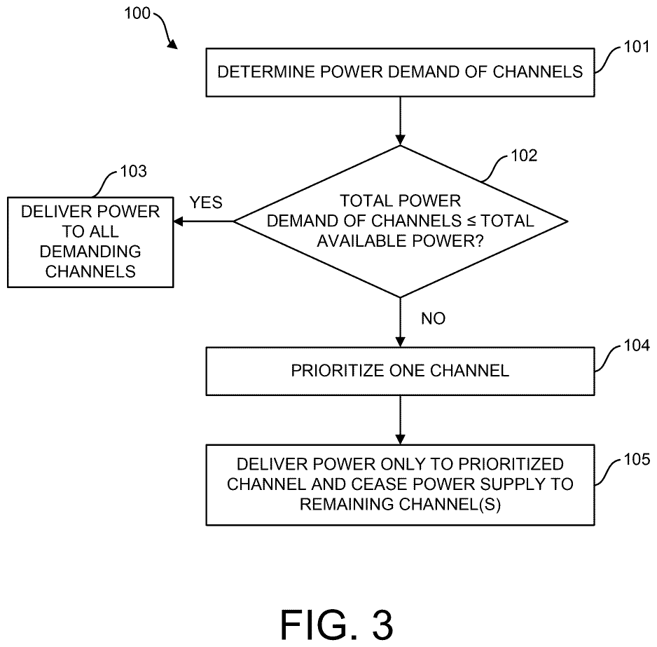

FIG. 3 is a flow diagram illustrating an embodiment of an example method 100 of allocating power delivery to or between one or more of the channels of the multichannel radiofrequency tissue ablation system 10 during the surgical procedure, thus providing a controlled operation of the RF generator during the procedure without experiencing unexpected and undesirable power interruptions to the two or more RF probes 70, 80. In accordance with the embodiments of the present invention, the power delivery allocation method 100 advantageously does not require user intervention to operate; however, in some embodiments, an output can be generated for display (e.g., via the user interface and display element 30 of the generator 20) sufficient to notify a user or operator of the activation status of the generator 20 and RF probes and to provide status updates for one or more associated parameters and components of the system 10, which are involved in the method 100. The method 100 comprises executing by the one or more processors 26 of the controller 24 program instructions stored in the memory element 28.

The method 100 of the present invention starts with determining by the controller 24 a power demand required by each of the channels during the surgical procedure, step 101. If the total power demand required by the RF probes 70, 80 does not exceed the total available power of the RF generator 20, step 102, power is delivered from the generator 20 to all the power demanding channels, step 103. If the total power demand required by the RF probes 70, 80 exceeds the total available power of the RF generator 20, step 102, the controller 24 allocates the radiofrequency power from the generator 20 to one of the channels. The power allocation to one of the channels can be implemented by prioritizing power delivery to a preselected one of the channels, step 104, and then delivering power only to the prioritized channel while ceasing power supply to the remaining one or more channels during the surgical procedure, step 105. If the total power demand required by the RF probes 70, 80 exceeds the total available power of the radiofrequency generator 20, step 102, the controller 24 allocates the radiofrequency power from the generator 20 to one of the channels. The power allocation to the channels can be based on a comparison of their respective remaining surgical times. For example, power may be allocated to the channel with the least remaining surgical time to enable it to complete treatment first. Alternatively, power could be allocated to the channel with the most remaining surgical time to ensure it can continue progressing. In general, the remaining surgical time information provides a basis for the controller to intelligently distribute limited power among the channels.

In a particular embodiment of the method 100 of the present invention, where the tissue ablation system 10 includes the first RF probe 70 and the second RF probe 80, the controller 24 can determine a power demand required by each of the first channel and the second channel during the surgical procedure, and when a total power demand required by the first and second RF probes 70, 80 via the first and second channels during the surgical procedure exceeds a total available power of the radiofrequency generator, the controller 24 can allocate the radiofrequency power from the generator 20 to one of the first and second channels. The power allocation technique can be implemented by prioritizing power delivery to a preselected one of the first and second channels and then delivering power only to the prioritized channel while ceasing or pausing power supply to the remaining one of the first and second channels during the surgical procedure.

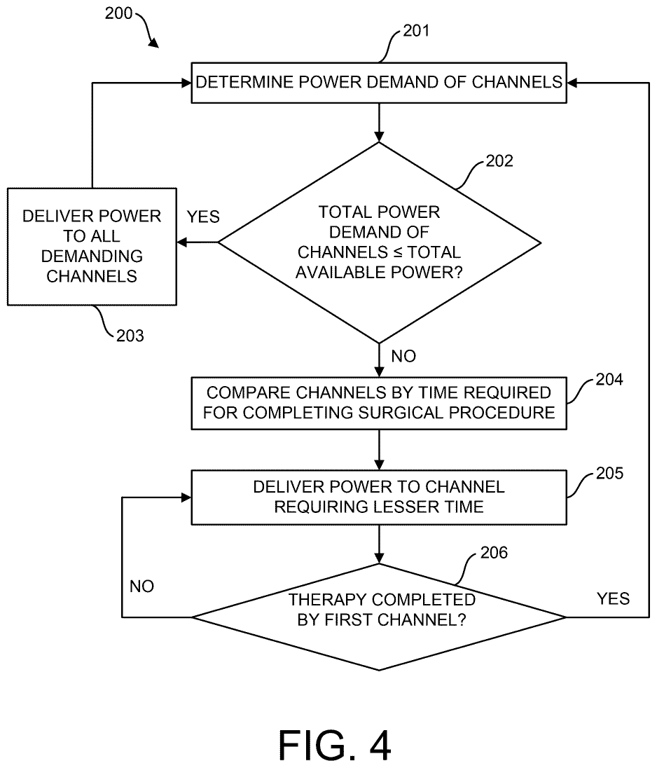

FIG. 4 is a schematic flow chart diagram illustrating a method 200 of allocating power delivery to the channels of the multichannel radiofrequency tissue ablation system 10 during a surgical procedure, which may be a particular embodiment of the method 100 illustrated in FIG. 3. In this embodiment, the surgical procedure has a particular surgical time associated with or allocated to each channel, and the amount of time remaining is set as a criteria for prioritizing power delivery to the first and second channels.

Specifically, the method 200 starts with the controller 24 determining a power demand or power amount required by each of the channels during the surgical procedure, step 201. If the total power demand or amount required by the RF probes 70, 80 does not exceed the total available power of the generator 20, step 202, power is delivered from the generator 20 to all the power demanding channels, step 203. If the total power demand required by the RF probes 70, 80 exceeds the total available power of the radiofrequency generator 20, the controller 24 compares the channels by time remaining in the surgical procedure associated with each channel, step 204.

The radiofrequency power is then generated by and delivered from the generator 20 to the channel having a lesser amount of time remaining in the surgical procedure relative to the other channels, step 205. The controller 24 can determine the proper channel by comparing the time remaining in the surgical procedures associated with the channel and then identify the channel with the least amount of time remaining in the surgical procedure. If the surgical procedure is not completed by the prioritized channel, step 206, the RF power from the generator 20 is continued to be delivered to the active prioritized channel, step 205. If the surgical procedure is completed by the prioritized channel, step 206, the controller 24 can then determine a power demand required by each of the remaining channels and then repeat the above-described steps 202-206 in respect of the remaining channels, as long as there is available power for one or more remaining channels to complete therapy.

In a particular embodiment of the method 200, when the tissue ablation system 10 comprises two RF probes 70, 80 (e.g., the first RF probe 70 and the second RF probe 80), the surgical procedure has a first surgical time associated with the first channel and a second surgical time associated with the second channel. The controller 24 then executes selected program instructions to compare a first time remaining in the first surgical procedure associated with the first channel with a second time remaining in the second surgical procedure associated with the second channel. Based on this comparison, the controller 24 can then determine which channel is to receive radiofrequency power from the RF generator 20. Specifically, the controller 24 determines which one of the first channel and the second channel having a lesser amount of time remaining in the surgical procedure, and then the generator 20 supplies power to the channel having the lesser amount of time remaining in the surgical procedure.

In some embodiments of the method 200, the controller 24 can be configured to order the channels based on the amount of time remaining in the surgical procedure, as a result of the comparison performed at step 204. In this embodiment, the delivery of radiofrequency power is initiated to each next channel in the channel order if the previous channel finishes therapy and the total power availability allows power to be supplied to the next channel in the channel order.

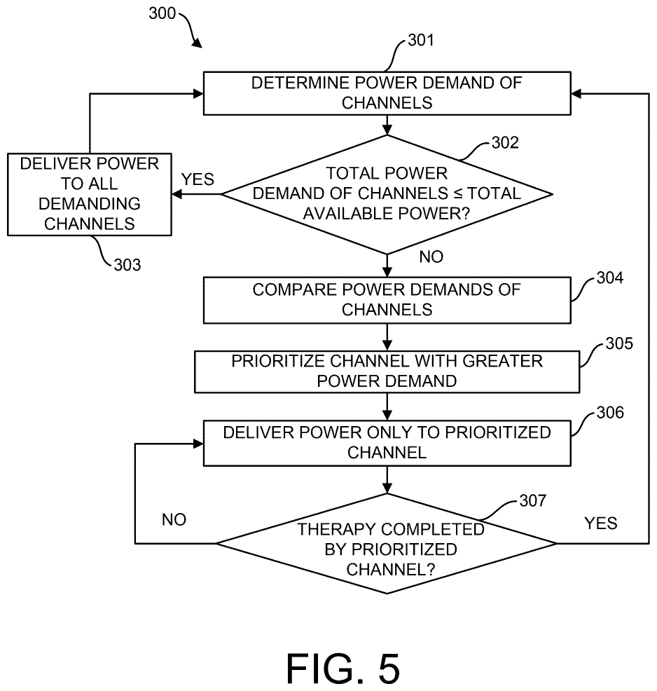

FIG. 5 is a schematic flow chart diagram illustrating a method 300 of allocating power delivery to the channels of the multichannel radiofrequency tissue ablation system 10 during a surgical procedure, which may be a particular embodiment of the method 100 of FIG. 3. In this embodiment, the surgical procedure has a total power demand associated therewith and each of the channels has a particular power demand associated therewith during the surgical procedure. The power demand parameter can be used to prioritize power delivery to the channels.

With reference to step 301, the method 300 starts with the controller 24 determining a power demand required by each of the channels during the surgical procedure. If the total power demand required by the RF probes 70, 80 does not exceed the total available power of the generator 20, step 302, the radiofrequency power can be delivered from the generator 20 to all of the power demanding channels, step 303. If the total power demand required by the RF probes 70, 80 exceeds the total available power of the radiofrequency generator 20, step 302, the controller 24 compares the power demands associated with each channel to each other during the surgical procedure, step 304.

The controller 24 then determines that the channel with the greater power demand, as a result of the comparison, is prioritized, step 305, and the radiofrequency power is delivered from the generator 20 only to the prioritized channel, step 306. If the surgical procedure is not completed by the prioritized channel (at the Decision Block 307), the power is continued to be delivered to the prioritized channel (Block 306). If the surgical procedure is completed by the prioritized channel, step 307, the method 300 may return to step 301 and repeat the above-described steps.

In particular, the controller 24 can reassess the power demands of the channels once the prioritized channel completes the associated surgical procedure, and if the power demand of any one of the channels exceeds the total available power of the RF generator 20, the controller 24 then resets the channel priority to the channel having a power demand that is less than or equal to the total available power of the generator 20. The new determination performed by the controller 24 forms a new prioritized channel. The generator 20 then delivers the radiofrequency power to the new prioritized channel while pausing or disallowing radiofrequency power delivery to the remaining one or more channels, until the new prioritized channel completes therapy.

In a particular embodiment of the method 300, when the tissue ablation system 10 includes multiple RF probes 70, 80 (e.g., the first radiofrequency probe 70 and the second RF probe 80), the first channel has a first power demand associated therewith during the associated surgical procedure and the second channel has a second power demand associated therewith during the associated surgical procedure. The controller then compares the first power demand of the first channel with the second power demand of the second channel, and then radiofrequency power from the RF generator 20 is supplied to the channel (e.g., prioritized channel) of the first and second channels having the greater power demand, in order to complete the surgical procedure.

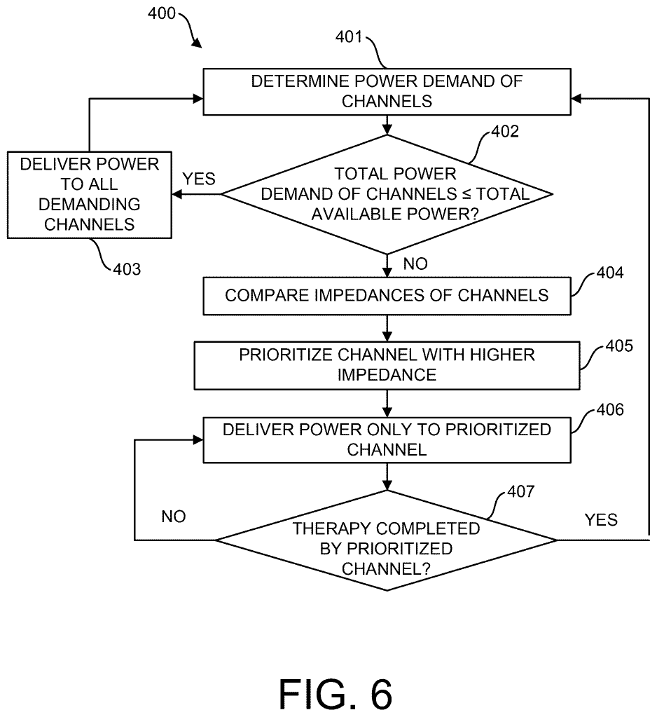

FIG. 6 is a schematic flow chart diagram illustrating a method 400 of allocating power delivery to the channels of the multichannel radiofrequency tissue ablation system 10 during a surgical procedure, which may be a particular embodiment of the method 100 of FIG. 3. In this embodiment, the impedances of the channels are set as power demand predictors. This approach assumes that the channels with higher impedances require higher power levels to achieve similar temperature profiles than those with lower impedances.

The illustrated method 400 starts with the controller 24 determining a power demand required by each of the channels during the surgical procedure, step 401. If the total power demand required by the channels (e.g., RF probes 70, 80) does not exceed the total available power of the generator 20, step 402, the power is delivered from the generator 20 to all of the power demanding channels, step 403. If the total power demand required by the RF probes 70, 80 (e.g., first and second channels) exceeds the total available power of the radiofrequency generator 20, the controller 24 then determines and compares the impedances of the channels, step 404.

According to method 400, the channel with the higher impedance, as a result of the comparison, is prioritized, step 406, and the radiofrequency power is delivered from the generator 20 only to the prioritized channel. If the surgical procedure is not completed by the prioritized channel, step 406, the generator 20 continues to deliver power to the prioritized channel, step 406. If the surgical procedure is completed by the prioritized channel, step 407, then the method 400 can return to step 401 and repeat the above-described steps in respect of the remaining channels if there is power available to be provided thereto.

In a particular embodiment of the method 400, when the tissue ablation system 10 includes multiple RF probes 70, 80 (e.g., the first radiofrequency probe 70 and the second RF probe 80), the controller 24 determines a first impedance associated with the first channel and a second impedance associated with the second channel, and then compares the first impedance with the second impedance. The generator 20 provides radiofrequency power to the channel having the higher impedance amount.

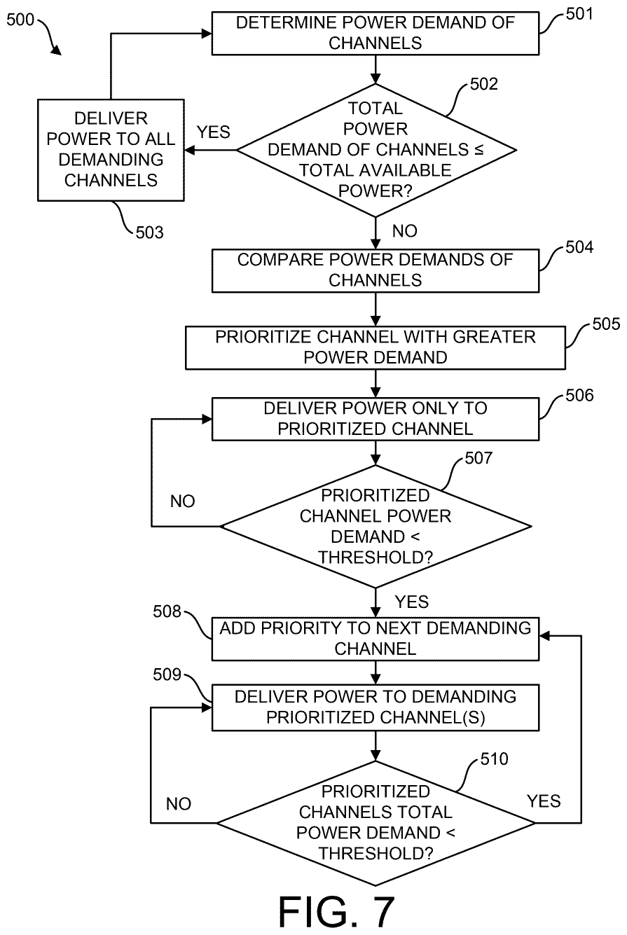

FIG. 7 is a schematic flow chart diagram illustrating another embodiment of a method 500 of allocating power to the channels of the multichannel radiofrequency tissue ablation system 10 during a surgical procedure. In this embodiment, the surgical procedure has a total power demand associated therewith and each of the channels has a particular power demand associated therewith during the surgical procedure. The power demand parameters are set as criteria for prioritizing power delivery to or among the channels.

The illustrated method 500 starts with the controller 24 determining a power demand required by each of the channels during the surgical procedure, step 501. If the total power demand required by the channels (e.g., the RF probes 70, 80) does not exceed the total available power of the radiofrequency generator 20, step 502, then the radiofrequency power is delivered from the generator 20 to all of the power demanding channels, step 503. If the total power demand required by the channels exceeds the total available power of the radiofrequency generator 20, step 502, then the controller 24 compares the power demands of each channel with reach other during the surgical procedure. step 504.

The controller 24 then prioritizes for power delivery the channel with the greater power demand, step 506. The radiofrequency power is delivered from the generator 20 only to the prioritized channel. If the power demand, such as the average power demand, of the prioritized channel is above or greater than a preset power demand threshold, step 507, the radiofrequency power is continued to be delivered to the prioritized channel, step 506, and the controller 24 disables an ability to provide radiofrequency power from the generator 20 to the remaining channels. If the power demand of the prioritized channel is less than the preset power demand threshold, step 507, which can occur if the active channel finishes therapy, and/or the power becomes available, then channel priority is assigned to one of the remaining channels, step 508, and power is provided to the newly assigned prioritized channel, step 509. If the power demand of the prioritized channel is not less than the preset threshold, step 510, the power is continued to be delivered to the prioritized channel, step 509. If the power demand of the prioritized channel is less than the preset threshold, step 510, then priority is assigned to the next one of the remaining channels, step 508, and power is provided to the new remaining prioritized channel, step 509. The steps 508-510 can be repeated as long as there are power demanding channels and power is available for the same.

Any methods described herein may be embodied in, and partially or fully automated via, software code modules (e.g., in the form of an algorithm or machine readable instructions) stored in a memory element such as a tangible, non-transitory computer-readable medium executed by one or more processors or other computing devices. The software may be downloaded to a processor in electronic form. In embodiments involving multiple processors, the processors may operate in parallel to form a parallel processing system in which a process is split into parts that execute simultaneously on different processors of the ablation system. The methods may be executed on the computing devices in response to execution of software instructions or other executable machine-readable code read from the memory or tangible computer readable medium. A tangible computer readable medium is a data storage device that can store data that is readable by a computer system. Examples of computer readable mediums include read-only memory (e.g., ROM or PROM, EEPROM), random-access memory, other volatile or non-volatile memory devices, CD-ROMs, magnetic tape, flash drives, and optical data storage devices.

As will be appreciated by a person of ordinary skill in the art, computer-executable instructions stored in tangible computer storage media define specific functions to be performed by computer hardware such as computer processors. In general, in such an implementation, the computer-executable instructions are loaded into memory accessible by at least one computer processor (for example, a programmable microprocessor or microcontroller or an application specific integrated circuit). The at least one computer processor then executes the instructions, causing computer hardware to perform the specific functions defined by the computer-executable instructions. As will be appreciated by a person of ordinary skill in the art, computer execution of computer-executable instructions is equivalent to the performance of the same functions by electronic hardware that includes hardware circuits that are hardwired to perform the specific functions. As such, while embodiments illustrated herein are typically implemented as some combination of computer hardware and computer-executable instructions, the embodiments illustrated herein could also be implemented as one or more electronic circuits hardwired to perform the specific functions illustrated herein.

Although certain embodiments and examples have been described herein, aspects of the methods and devices shown and described in the present disclosure may be differently combined and/or modified to form still further embodiments. Optional features of various device and system embodiments may be included in some embodiments and not in others. Additionally, the methods described herein may be practiced using any device suitable for performing the recited steps. Further, the disclosure (including the figures) herein of any particular feature, aspect, method, property, characteristic, quality, attribute, element, or the like in connection with various embodiments can be used in all other embodiments set forth herein. Any section headings used herein are merely provided to enhance readability and are not intended to limit the scope of the embodiments disclosed in a particular section to the features or elements disclosed in that section.

While the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited and in other alternative embodiments one or more method steps may be skipped altogether.

Various embodiments of the disclosure have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 70 to 115 degrees should be considered to have specifically disclosed subranges as well as individual numbers within that range, for example, 70, 70.5, 80, 115, and any whole and partial increments therebetween. Language such as “at least,” “greater than,” “less than,” “between,” and the like includes the number recited. Numbers preceded by a term such as “about” (in the meaning of “approximately”) include the recited numbers. For example, “between about 5 W and about 30 W” includes 5 W and 30 W. As used herein in the specification and claims, including as used in the examples and unless otherwise expressly specified, all numbers may be read as if prefaced by the word “about” even if the term does not expressly appear. The phrase “about” may be used when describing magnitude to indicate that the value described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “70” is disclosed, then “about 70” is also disclosed. Any numerical range recited herein is intended to include all sub-ranges subsumed therein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

What is claimed is:

1. A tissue ablation system, comprising:
a radiofrequency generator having a power source and a controller operatively coupled to the power source and having a processor and a memory element,
a first radiofrequency probe having at least one electrode and electrically coupled to the radiofrequency generator to form a first channel, and
a second radiofrequency probe having at least one electrode and electrically coupled to the radiofrequency generator to form a second channel,
wherein the power source is configured to deliver radiofrequency power to the one or more electrodes of each of the first and second radiofrequency probes, the radiofrequency power being sufficient to provide controlled heating of tissue surrounding the one or more electrodes during a surgical procedure,
wherein the processor is configured to execute program instructions stored in the memory element such that, when executed:
cause the controller to determine a power demand required by each of the first channel and the second channel during the surgical procedure, and
when a total power demand required by the first and second radiofrequency probes via the first and second channels during the surgical procedure exceeds a total available power of the radiofrequency generator, cause the controller to allocate the radiofrequency power from the radiofrequency generator to one of the first and second channels.

2. The system of claim 1, wherein the processor is further configured by the program instructions to cause the controller to:
prioritize providing the radiofrequency power from the radiofrequency generator to a preselected one of the first and second channels, and
cease power supplied to a remaining one of the first and second channels during the surgical procedure.

3. The system of claim 1, wherein the surgical procedure has a first surgical time associated with the first channel and a second surgical time associated with the second channel, and wherein the processor is further configured by the program instructions to cause the controller to:
compare a first time remaining of the first surgical time in the surgical procedure associated with the first channel with a second time remaining of the second surgical time in the surgical procedure associated with the second channel, and
based on the comparison, provide the radiofrequency power from the radiofrequency generator to one of the first channel and the second channel having a lesser amount of time remaining in the surgical procedure.

4. The system of claim 3, wherein the processor is further configured by the program instructions to cause the controller to order the first and second channels based on the amount of time remaining in the surgical procedure.

5. The system of claim 1, wherein the surgical procedure has a total power demand associated therewith and wherein the first channel has a first power demand associated therewith during the surgical procedure and the second channel has a second power demand associated therewith during the surgical procedure, wherein the processor is further configured by the program instructions to cause the controller to:
compare the first power demand with the second power demand, and
provide the radiofrequency power from the radiofrequency generator to one of the first and second channels having the greater power demand to complete the surgical procedure to form a prioritized channel.

6. The system of claim 5, wherein the processor is further configured by the program instructions to cause the controller to:
determine an average power demand of the prioritized channel, and
if the average power demand of the prioritized channel is above a power demand threshold, then disable an ability to provide radiofrequency power from the radiofrequency generator to a remaining one of the first and second channels.

7. The system of claim 5, wherein the processor is further configured by the program instructions to cause the controller to:
reassess the first and second power demands of the first and second channels once the prioritized channel completes the surgical procedure, and
if the power demand of any one of the first and second channels exceeds the total available power of the radiofrequency generator:
reset priority to the channel having a power demand that is less than or equal to the total available power of the radiofrequency generator to form a new prioritized channel, and
cause the power source to deliver radiofrequency power to the new prioritized channel while pausing or disallowing radiofrequency power delivery to the remaining one of the first and second channels until the new prioritized channel completes therapy.

8. The system of claim 1, wherein the processor is further configured by the program instructions to cause the controller to:
determine a first impedance associated with the first channel and a second impedance associated with the second channel, and
compare the first impedance with the second impedance, and
provide the radiofrequency power from the radiofrequency generator to one of the first and second channels having a higher impedance amount.

9. The system of claim 1, wherein the processor is further configured by the program instructions to cause the controller to order the first and second channels based on time remaining for completing the surgical procedure.

10. A multichannel radiofrequency tissue ablation system, comprising
a radiofrequency generator having a power source and a controller operatively coupled to the power source and having a processor and a memory element,
a first radiofrequency probe having at least one electrode and is electrically coupled to the radiofrequency generator to form a first channel, and
a second radiofrequency probe having at least one electrode and is electrically coupled to the radiofrequency generator to form a second channel,
wherein the power source is configured to deliver radiofrequency power to the one or more electrodes of each of the first and second radiofrequency probes, the radiofrequency power being sufficient to provide controlled heating of tissue surrounding the one or more electrodes during a surgical procedure,
wherein the controller includes a power determination and allocation unit to determine a power demand required by each of the first channel and the second channel during the surgical procedure, and when a total power demand required by the first and second radiofrequency probes via the first and second channels during the surgical procedure exceeds a total available power of the radiofrequency generator, the power determination and allocation unit allocates the radiofrequency power from the radiofrequency generator to one of the first and second channels.

11. The system of claim 10, wherein the power determination and allocation unit is further configured to prioritize providing the radiofrequency power from the radiofrequency generator to a preselected one of the first and second channels, and cease power supplied by the radiofrequency generator to a remaining one of the first and second channels during the surgical procedure.

12. The system of claim 10, wherein the surgical procedure has a first surgical time associated with the first channel and a second surgical time associated with the second channel, and wherein the controller further comprises a comparison unit for comparing a first time remaining of the first surgical time in the surgical procedure associated with the first channel with a second time remaining of the second surgical time in the surgical procedure associated with the second channel, and wherein, based on the comparison, the power determination and allocation unit is configured to enable the radiofrequency generator to supply the radiofrequency power to one of the first channel and the second channel.

13. The system of claim 10, wherein the surgical procedure has a total power demand associated therewith and wherein the first channel has a first power demand associated therewith during the surgical procedure and the second channel has a second power demand associated therewith during the surgical procedure, and wherein the controller further comprises a comparison unit for comparing the first power demand with the second power demand, and wherein, based on the comparison, the power determination and allocation unit is configured to enable the radiofrequency generator to supply the radiofrequency power to one of the first and second channels having the greater power demand to complete the surgical procedure to form a prioritized channel.

14. The system of claim 13, wherein the power determination and allocation unit is further configured to determine an average power demand of the prioritized channel, and if the average power demand of the prioritized channel is above a power demand threshold, then disable an ability to for the radiofrequency generator to provide radiofrequency power to a remaining one of the first and second channels.

15. The system of claim 10, wherein the controller further comprises an impedance determination unit to determine a first impedance associated with the first channel and a second impedance associated with the second channel, wherein the comparison unit is configured to compare the first impedance with the second impedance, and wherein, based on the comparison, the power determination and allocation unit is configured to enable the radiofrequency generator to provide the radiofrequency power to one of the first and second channels having a higher impedance amount.

16. A computer-implemented method of allocating power delivery in a tissue ablation system, the system comprising: a radiofrequency generator having a power source and a controller operatively coupled to the power source and having a processor and a memory element, a first radiofrequency probe having at least one electrode and electrically coupled to the radiofrequency generator to form a first channel, and a second radiofrequency probe having at least one electrode and electrically coupled to the radiofrequency generator to form a second channel, the power source configured to deliver radiofrequency power to the one or more electrodes of each of the first and second radiofrequency probes, the radiofrequency power being sufficient to provide controlled heating of tissue surrounding the one or more electrodes during a surgical procedure, the method comprising executing via the processor program instructions stored in the memory element to:
cause the controller to determine a power demand required by each of the first channel and the second channel during the surgical procedure, and
when a total power demand required by the first and second radiofrequency probes via the first and second channels during the surgical procedure exceeds a total available power of the radiofrequency generator, cause the controller to allocate the radiofrequency power from the radiofrequency generator to one of the first and second channels.

17. The computer-implemented method of claim 16, wherein the surgical procedure has a first surgical time associated with the first channel and a second surgical time associated with the second channel, and wherein the method further comprises executing via the processor program instructions stored in the memory element to:
compare a first time remaining of the first surgical time in the surgical procedure associated with the first channel with a second time remaining of the second surgical time in the surgical procedure associated with the second channel, and
provide the radiofrequency power from the radiofrequency generator to one of the first channel and the second channel having a lesser amount of time remaining in the surgical procedure.

18. The computer-implemented method of claim 16, wherein the surgical procedure has a total power demand associated therewith and wherein the first channel has a first power demand associated therewith during the surgical procedure and the second channel has a second power demand associated therewith during the surgical procedure, and wherein the method further comprises executing via the processor program instructions stored in the memory element to:
compare the first power demand with the second power demand, and
provide the radiofrequency power from the radiofrequency generator to one of the first and second channels having the greater power demand to complete the surgical procedure to form a prioritized channel.

19. The computer-implemented method of claim 18, further comprising executing via the processor program instructions stored in the memory element to:
determine an average power demand of the prioritized channel, and
if the average power demand of the prioritized channel is above a power demand threshold, then disable an ability to for the radiofrequency generator to provide radiofrequency power to a remaining one of the first and second channels.

20. The computer-implemented method of claim 16, further comprising executing via the processor program instructions stored in the memory element to:
determine a first impedance associated with the first channel and a second impedance associated with the second channel, and
compare the first impedance with the second impedance, and
provide the radiofrequency power from the radiofrequency generator to one of the first and second channels having a higher impedance amount.
